(19) United States
(12) Patent Application Publication
(10) Pub. No.: US 2005/0227351 A1
(43) Pub. Date: Oct. 13, 2005

(54) Methods and compound protein molecules used for inducing cytotoxic cell-mediated immune response in a mammal against a pathogenic cell

(76) Inventor: Essam T. Awdalla, Raleigh, NC (US)

Correspondence Address: MACCORD MASON PLLC, 300 N. GREENE STREET, SUITE 1600, P. O. BOX 2974, GREENSBORO, NC, 27402, US

(21) Appl. No.: 10/820,569

(22) Filed: Apr. 8, 2004

(51) Int. Cl. C12Q 1/70; C12Q 1/68; G01N 33/53; A61K 39/395; A61K 39/40; A61K 39/42; C12N 5/06; C12N 5/16

(52) U.S. Cl. 435/345; 435/6; 435/7.1

(57) ABSTRACT

The present invention provides compound protein molecules for inducing cytotoxic cell-mediated immune response in a mammal against a pathogenic cell displaying an antigen on its outer surface. In a preferred embodiment, the compound protein molecule comprises in sequence: a foreign class I MHC molecule; a linking (Fab) antibody fragment; an alpha-helix shaped chain linker; and a (Fab) fragment of an antibody specific against the antigen displayed by the pathogenic cell. In another preferred embodiment, the compound protein molecule comprises in sequence: a foreign class I MHC molecule; a linking (Fab) antibody fragment; an alpha-helix shaped chain linker; a (Fab′2) fragment of an antibody specific against the antigen displayed by the pathogenic cell; an alpha-helix shaped chain linker; a linking (Fab) antibody fragment; and a foreign class I MHC molecule. Methods for inducing an immune response using these compound protein molecules are also disclosed.

### FIELD OF THE INVENTION

The present invention relates to methods and compound protein molecules used for inducing a cytotoxic cell-mediated immune response in a mammal against a pathogenic cell, and more particularly, to methods and compound protein molecules used for inducing eradicative immune responses against cancerous and virus-infected cells.

### BACKGROUND OF THE INVENTION

The immune system in man and other types of mammals is a complex of organs and various types of highly specialized cells that work together to clear pathogens from the body.

It is generally accepted that a cell-mediated immune response is induced when an antigen is presented by the major histocompatibility complex (MHC) molecules on the outer cell surface of Macrophages (which are one of the cell types forming the immune system), after being engulfed and processed by them. Cell mediated immune response either leads to the synthesis of antibodies specific against the antigen, which is referred to as “Humoral immune response”, or leads to the production of “sensitized” lymphocytes, which attack and destroy cells displaying the antigen on their outer surface, and which is referred to as “Cytotoxic cell-mediated immune response”. It is generally accepted that a “humoral immune response” is induced when the antigen is presented by MHC class II molecules on the outer surface of the Macrophages, while a “cytotoxic cell-mediated immune response” is induced when the antigen is presented by MHC class I molecules.

Foreign grafts rejection involves the induction of a “cytotoxic cell-mediated immune response”, and results from the recognition of the foreign class I MHC molecules on the outer surface of the cells of the graft as non-self antigens, as the MHC molecules of one individual are considered foreign by the cells of the immune system of another individual, except in the case of identical twins wherein exact matching between the MHC molecules occurs.

In case of tumors (and some types of viral infections), either a “humoral immune response” or a “cytotoxic cell-mediated immune response” may be elicited, according to the nature of the antigen(s) displayed by the cancerous cells (or the virus-infected cells) and identified by the cells of the individual's immune system. When a “cytotoxic cell-mediated immune response” is induced against the cancerous cells, it leads to their complete elimination from the body, with the whole process being unnoticed by the individual. On the other hand, when only a “humoral immune response” is induced, it usually fails to destroy the cancerous cells, with ultimate growth of the tumor within the body.

Prior art documents include components related to the field of the present invention but lack an integrated, combined solution that is provided by the present invention, including the following: truncated MHC complexes are prepared by the method described by Rhode, et al. U.S. Pat. No. 5,869,270; altered MHC determinant molecules are prepared by the method described by Mottez, et al. U.S. Pat. No. 6,011,146; preparation of antibody molecules such as described by Bjorklund U.S. Pat. No. 3,960,827; preparation of alpha-helix shaped chains such as described by Miura, et al. U.S. Pat. No. 6,124,429, and by Abraham, et al. U.S. Pat. No. 6,624,140; detecting the presence of a specific type of antigen in a sample such as described by Bolz, et al. U.S. Pat. No. 4,020,151, and by Pradelles U.S. Pat. No. 5,476,770; detecting antibodies as described by Weetall, et al. U.S. Pat. No. 4,024,235; examples of blocking groups and methods of making substrates containing blocking groups are described in Methods in Enzymology, Vol. 244, “Proteolytic Enzymes,” A. J. Barrett, Ed., Chapters 46, 47, and 48, (1994); and Green and Wuts, Protective Groups in Organic Synthesis, John Wiley and Sons, Pub., (1991); catalyses of the disulfide bond in the presence of low molecular weight thiol reagents in reduced and oxidized form, as described in Saxena et al., Biochemistry 9: 5015-5021 (1970).

Thus, there remains a need for a method for inducing an eradicative cytotoxic cell-mediated immune response in a mammal against a pathogenic cell displaying an antigen known to elicit a non-eradicative humoral immune response, and compound protein molecule therefor, as provided by the present invention.

### SUMMARY OF THE INVENTION

The present invention is directed to and provides, in one aspect of the invention, a method for inducing an eradicative cytotoxic cell-mediated immune response in a mammal against a pathogenic cell displaying an antigen known to elicit a non-eradicative humoral immune response. The present invention is further directed to and provides, in another aspect of the invention, a compound protein molecule, used for inducing a cytotoxic cell-mediated immune response in a mammal against a pathogenic cell displaying an antigen on its outer surface as set forth hereinbelow.

Accordingly, the outer surface of the pathogenic cell is tagged with a compound protein molecule including an antigenic molecule known to induce a cytotoxic cell-mediated immune response, i.e., a foreign class I MHC molecule. As used herein, a foreign class I MHC molecule refers to a class I MHC molecule recognized as non-self by the immune system of the mammal.

To enable fixing the foreign class I MHC molecule on the outer surface of the pathogenic cell, the compound protein molecule further includes an antibody molecule specific against the antigen displayed on the outer surface of the pathogenic cell, noting that the link between the foreign class I MHC molecule and the antibody molecule should provide acceptable functional orientation between the two molecules relative to one another, so that when the antibody molecule gets attached to its respective antigen on the outer surface of the pathogenic cell, the binding groove or cleft of the foreign class I MHC molecule will be facing relatively outwards, to enable its detection by the sensitized lymphocytes. The used antibody molecule is either a (Fab) fragment or a (Fab′2) fragment of a monoclonal antibody specific against the antigen displayed on the outer surface of the target pathogenic cell. Such antibody fragments and means for their preparation are well known to people experienced in the art. The foreign class I MHC molecule may be a full length MHC molecule, a single chain truncated MHC complex or an altered MHC determinant molecule. Full length MHC molecules are obtained from a suitable cell line or prepared by recognized recombinant DNA techniques, e.g. preparation of plasmid DNA, cleavage of DNA with restriction enzymes, ligation of DNA, transformation or transfection of a host, culturing of the host, and isolation and purification of the expressed fusion complex. Such procedures are generally known and disclosed e.g. in Sambrook et al., Molecular Cloning (2nd ed. 1989). Truncated MHC complexes are prepared by the method described by Rhode, et al. U.S. Pat. No. 5,869,270, herein incorporated by reference in its entirety.

Altered MHC determinant molecules are prepared by the method described by Mottez, et al. U.S. Pat. No. 6,011,146, herein incorporated by reference in its entirety.

The antibody molecules can be prepared using a wide variety of techniques known in the art including the use of hybridoma, recombinant, and phage display technologies, or a combination thereof. Non limiting example for such techniques is described by Bjorklund U.S. Pat. No. 3,960,827, herein incorporated by reference in its entirety. The prepared antibody molecules are either digested by the Papain enzyme to yield the (Fab) antibody fragments, or by the Pepsin enzyme to yield the (Fab′2) antibody fragments.

When (Fab) antibody fragments are used, the carboxy-terminal end of the heavy chain of each of the (Fab) antibody fragments is linked to the carboxy-terminal end of one of the used foreign class I MHC molecules, through intermediate linking means, as will be discussed in detail herein after. When (Fab′2) antibody fragments are used, the carboxy-terminal end of each of the two heavy chains of the (Fab′2) antibody fragments is linked to the carboxy-terminal end of one of the used foreign MHC molecules, through intermediate linking means, with the prepared compound protein molecule having two foreign class I MHC molecules, as will be discussed in detail herein after.

In a preferred embodiment, the intermediate linking means, used to link the carboxy-terminal ends of each of the used antibody fragments and the foreign class I MHC molecules to one another, is a protein complex comprising a (Fab) antibody fragment, which doesn't need to be the same type of antibody referred to herein before, and which is referred to herein after as the “linking (Fab) antibody fragment”; and an alpha-helix shaped chain linker. The light chains and the heavy chains of the linking (Fab) antibody fragment are prepared separately, preferably using recombinant DNA techniques referred to herein before. The alpha-helix shaped chain linker is also preferably prepared using recombinant DNA techniques. Non limiting examples for preparation of alpha-helix shaped chains are described by Miura, et al. U.S. Pat. No. 6,124,429, and by Abraham, et al. U.S. Pat. No. 6,624,140, herein incorporated by reference in their entirety.

Accordingly, the method provided in the present invention for inducing a cytotoxic cell-mediated immune response in a mammal against a pathogenic cell displaying an antigen on its outer surface comprises the steps of:
1) Specifying the type of the antigen displayed by the pathogenic cell:
   a) in case of cancerous cells: a sample of cancerous cells is obtained by fine needle aspiration biopsy, core needle biopsy, or vacuum-assisted biopsy techniques. Such techniques are well known to people experienced in the art. The type of antigen displayed by the cancerous cells is then specified by one of the methods known in the art for detecting the presence of a specific type of antigen in a sample. Non limiting examples of such methods are described by Bolz, et al. U.S. Pat. No. 4,020,151, and by Pradelles U.S. Pat. No. 5,476,770, herein incorporated by reference in their entirety.
   b) In case of virus-infected cells, the type of antigen displayed is specified indirectly by specifying the type of the formed antibody present in the serum of the infected mammal against it. Non limiting examples for the methods used for detecting such antibodies is described by Weetall, et al. U.S. Pat. No. 4,024,235, herein incorporated by reference in its entirety.
2) Providing a supply of monoclonal antibodies specific against the specified antigen displayed by the pathogenic cell, using one of the techniques known in the art and referred to herein before.
3) Blocking the carboxy-terminal ends of both the light and heavy chains of the monoclonal antibodies of step 2, using one of the blocking groups known in the art. Examples of blocking groups and methods of making substrates containing blocking groups are described in Methods in Enzymology, Vol. 244, “Proteolytic Enzymes,” A. J. Barrett, Ed., Chapters 46, 47, and 48, (1994); and Green and Wuts, Protective Groups in Organic Synthesis, John Wiley and Sons, Pub., (1991), herein incorporated by reference in their entirety. The blocked antibodies are then digested by either the Papain enzyme to yield (Fab) antibody fragments with blocked carboxy-terminal ends of only their light chains (as the parts having the blocked carboxy-terminal ends of the heavy chains will be removed by digestion), or by the Pepsin enzyme to yield (Fab′2) antibody fragments with blocked carboxy-terminal ends of only their light chains, as described herein before.
4) Providing a supply of alpha-helix shaped chain linkers using one of the techniques referred to herein before.
5) Linking the amino-terminal end of the alpha-helix shaped chain linkers of step 4 to the carboxy-terminal ends of the heavy chains of the antibody fragments of step 3 (either the (Fab) fragments or the (Fab′2) fragments) using “Solid phase peptide synthesis” (SPPS) technique well known to people experienced in the art.
6) Providing a supply of light chains (or heavy chains) of linking (Fab) antibody fragments using one of the techniques referred to herein before.
7) Linking the amino-terminal ends of the light chains (or heavy chains) of the linking (Fab) antibody fragments of step 6 to the carboxy-terminal ends of the alpha-helix shaped chain linkers included in the complexes prepared in step 5, using SPPS technique.
8) Providing a supply of foreign class I MHC molecules using one of the techniques referred to herein before.
9) Providing a supply of heavy chains (or light chains) of linking (Fab) antibody fragments using one of the techniques referred to herein before.
10) Linking the amino-terminal ends of the heavy chains (or the light chains) of the linking (Fab) antibody fragments of step 9 to the carboxy-terminal ends of the foreign class I MHC molecules of step 8, using SPPS technique.
11) Joining the light chains (or the heavy chains) of the linking (Fab) antibody fragments included in the complexes prepared in step 7 with the heavy chains (or the light chains) of the linking (Fab) antibody fragments included in the complexes prepared in step 10 by disulfide bond formation using techniques well known to people experienced in the art, e.g. catalyses of the disulfide bond in the presence of low molecular weight thiol reagents in reduced and oxidized form, as described in Saxena et al., Biochemistry 9: 5015-5021 (1970), incorporated herein by reference in its entirety.
12) Sensitizing the mammal against the foreign class I MHC molecules provided in step 8 using well known immunization techniques, e.g., subcutaneous injection of the foreign MHC molecules, either alone or mixed with a suitable immune adjuvant.

13) Administering the compound protein molecules prepared in step 11 to the mammal via a suitable route.

The suitable route for administering the said compound protein molecules depends on the type of the pathogenic cell, so,
a) for cancerous cells the compound protein molecules are administered by either intravenous injection (to induce a cytotoxic cell-mediated immune response against any 2ry deposits spreading through the blood stream), by spraying into an open wound during an operation conducted to excise the main bulk of the tumor (to induce a cytotoxic cell-mediated immune response against any 2ry deposits present within the operation field, or spreading to nearby tissues through the lymphatic system), or preferably via both routes.
b) for virus-infected cells the compound protein molecules are administered by intravenous injection, preferably after temporary suppression of the reproduction cycle of the pathogenic virus by a suitable drug, to decrease the level of anti-viral antibodies formed within the blood of the mammal, which would otherwise compete with the administered compound protein molecules for binding with the antigens displayed by the virus-infected cells. Non limiting examples of such drugs are Nucleoside Reverse Transcriptase Inhibitors (NRTIs) and non-nucleoside reverse transcriptase inhibitors (NNRTIs), which are well known to people experienced in the art. The present invention also provides compound protein molecules to be used for inducing a cytotoxic cell-mediated immune response in a mammal against a pathogenic cell displaying an antigen on its outer surface, which are prepared using the techniques referred to herein before. In a preferred embodiment, the compound protein molecule comprises in sequence: 1) a foreign class I MHC molecule; 2) a linking (Fab) antibody fragment; 3) an alpha-helix shaped chain linker; and 4) a (Fab) fragment of an antibody specific against the antigen displayed on the outer surface of the pathogenic cell.

In another preferred embodiment, the compound protein molecule comprises in sequence: 1) a foreign class I MHC molecule; 2) a linking (Fab) antibody fragment; 3) an alpha-helix shaped chain linker; 4) a (Fab′2) fragment of an antibody specific against the antigen displayed on the outer surface of the pathogenic cell; 5) an alpha-helix shaped chain linker; 6) a linking (Fab) antibody fragment; and 7) a foreign class I MHC molecule.

These and other aspects of the present invention will become apparent to those skilled in the art after a reading of the following description of the preferred embodiment when considered with the drawings and the claims.

### BRIEF DESCRIPTION OF THE DRAWINGS

The description of the features of the present invention will be more fully appreciated by reference to the following detailed description of the exemplary embodiments in accordance with the accompanying drawings, wherein:

FIG. 1 is a schematic representation of a preferred embodiment of the compound protein molecules provided in the present invention, showing the orientation of its components relative to one another.

FIG. 2 is a schematic representation showing the steps of preparing the compound protein molecule of FIG. 1.

FIG. 3 is a schematic representation of another preferred embodiment of the compound protein molecules provided in the present invention, showing the orientation of its components relative to one another.

FIG. 4 is a schematic representation showing the steps of preparing the compound protein molecule of FIG. 3.

### DETAILED DESCRIPTION OF THE PREFERRED EMBODIMENTS

The present invention provides a method for inducing an eradicative cytotoxic cell-mediated immune response in a mammal against a pathogenic cell displaying an antigen known to elicit a non-eradicative humoral immune response.

Accordingly, the outer surface of the pathogenic cell is tagged with a compound protein molecule including an antigenic molecule known to induce a cytotoxic cell-mediated immune response, i.e., a foreign class I MHC molecule. As used herein, a foreign class I MHC molecule refers to a class I MHC molecule recognized as non-self by the immune system of the mammal. To enable fixing the foreign class I MHC molecule on the outer surface of the pathogenic cell, the compound protein molecule further includes an antibody molecule specific against the antigen displayed on the outer surface of the pathogenic cell, noting that the link between the foreign class I MHC molecule and the antibody molecule should provide acceptable functional orientation between the two molecules relative to one another, so that when the antibody molecule gets attached to its respective antigen on the outer surface of the pathogenic cell, the binding groove or cleft of the foreign class I MHC molecule will be facing relatively outwards, to enable its detection by the sensitized lymphocytes. The used antibody molecule is either a (Fab) fragment or a (Fab′2) fragment of a monoclonal antibody specific against the antigen displayed on the outer surface of the target pathogenic cell. Such antibody fragments and means for their preparation are well known to people experienced in the art. The foreign class I MHC molecule may be a full length MHC molecule, a single chain truncated MHC complex or an altered MHC determinant molecule. Full length MHC molecules are obtained from a suitable cell line or prepared by recognized recombinant DNA techniques, e.g. preparation of plasmid DNA, cleavage of DNA with restriction enzymes, ligation of DNA, transformation or transfection of a host, culturing of the host, and isolation and purification of the expressed fusion complex. Such procedures are generally known and disclosed e.g. in Sambrook et al., Molecular Cloning (2nd ed. 1989). Truncated MHC complexes are prepared by the method described by Rhode, et al. U.S. Pat. No. 5,869,270, herein incorporated by reference in its entirety. Altered MHC determinant molecules are prepared by the method described by Mottez, et al. U.S. Pat. No. 6,011,146, herein incorporated by reference in its entirety.

The antibody molecules can be prepared using a wide variety of techniques known in the art including the use of hybridoma, recombinant, and phage display technologies, or a combination thereof. Non limiting example for such techniques is described by Bjorklund U.S. Pat. No. 3,960,827, herein incorporated by reference in its entirety. The prepared antibody molecules are either digested by the Papain enzyme to yield the (Fab) antibody fragments, or by the Pepsin enzyme to yield the (Fab′2) antibody fragments. When (Fab) antibody fragments are used, the carboxy-terminal end of the heavy chain of each of the (Fab) antibody fragments is linked to the carboxy-terminal end of one of the used foreign class I MHC molecules, through intermediate linking means, as will be discussed in detail herein after. When (Fab′2) antibody fragments are used, the carboxy-terminal end of each of the two heavy chains of the (Fab′2) antibody fragments is linked to the carboxy-terminal end of one of the used foreign MHC molecules, through intermediate linking means, with the prepared compound protein molecule having two foreign class I MHC molecules, as will be discussed in detail herein after. In a preferred embodiment, the intermediate linking means, used to link the carboxy-terminal ends of each of the used antibody fragments and the foreign class I MHC molecules to one another, is a protein complex comprising a (Fab) antibody fragment, which doesn't need to be the same type of antibody referred to herein before, and which is referred to herein after as the “linking (Fab) antibody fragment”; and an alpha-helix shaped chain linker. The light chains and the heavy chains of the linking (Fab) antibody fragment are prepared separately, preferably using recombinant DNA techniques referred to herein before. The alpha-helix shaped chain linker is also preferably prepared using recombinant DNA techniques. Non limiting examples for preparation of alpha-helix shaped chains are described by Miura, et al. U.S. Pat. No. 6,124,429, and by Abraham, et al. U.S. Pat. No. 6,624,140, herein incorporated by reference in its entirety.

Accordingly, the method provided in the present invention for inducing a cytotoxic cell-mediated immune response in a mammal against a pathogenic cell displaying an antigen on its outer surface includes the steps of:
1) Specifying the type of the antigen displayed by the pathogenic cell:
   a) in case of cancerous cells: a sample of cancerous cells is obtained by fine needle aspiration biopsy, core needle biopsy, or vacuum-assisted biopsy techniques. Such techniques are well known to people experienced in the art. The type of antigen displayed by the cancerous cells is then specified by one of the methods known in the art for detecting the presence of a specific type of antigen in a sample. Non limiting examples of such methods are described by Bolz, et al. U.S. Pat. No. 4,020,151, and by Pradelles U.S. Pat. No. 5,476,770, herein incorporated by reference in its entirety.
   b) In case of virus-infected cells, the type of antigen displayed is specified indirectly by specifying the type of the formed antibody present in the serum of the infected mammal against it. Non limiting examples for the methods used for detecting such antibodies is described by Weetall, et al. U.S. Pat. No. 4,024,235, herein incorporated by reference in its entirety.
2) Providing a supply of monoclonal antibodies specific against the specified antigen displayed by the pathogenic cell, using one of the techniques known in the art and referred to herein before.
3) Blocking the carboxy-terminal ends of both the light and heavy chains of the monoclonal antibodies of step 2, using one of the blocking groups known in the art. Examples of blocking groups and methods of making substrates containing blocking groups are described in Methods in Enzymology, Vol. 244, “Proteolytic Enzymes,” A. J. Barrett, Ed., Chapters 46, 47, and 48, (1994); and Green and Wuts, Protective Groups in Organic Synthesis, John Wiley and Sons, Pub., (1991), herein incorporated by reference in its entirety. The blocked antibodies are then digested by either the Papain enzyme to yield (Fab) antibody fragments with blocked carboxy-terminal ends of only their light chains (as the parts having the blocked carboxy-terminal ends of the heavy chains will be removed by digestion), or by the Pepsin enzyme to yield (Fab′2) antibody fragments with blocked carboxy-terminal ends of only their light chains, as described herein before.
4) Providing a supply of alpha-helix shaped chain linkers using one of the techniques referred to herein before.
5) Linking the amino-terminal end of the alpha-helix shaped chain linkers of step 4 to the carboxy-terminal ends of the heavy chains of the antibody fragments of step 3 (either the (Fab) fragments or the (Fab′2) fragments) using “Solid phase peptide synthesis” (SPPS) technique well known to people experienced in the art.
6) Providing a supply of light chains (or heavy chains) of linking (Fab) antibody fragments using one of the techniques referred to herein before.
7) Linking the amino-terminal ends of the light chains (or heavy chains) of the linking (Fab) antibody fragments of step 6 to the carboxy-terminal ends of the alpha-helix shaped chain linkers included in the complexes prepared in step 5, using SPPS technique.
8) Providing a supply of foreign class I MHC molecules using one of the techniques referred to herein before.
9) Providing a supply of heavy chains (or light chains) of linking (Fab) antibody fragments using one of the techniques referred to herein before.
10) Linking the amino-terminal ends of the heavy chains (or the light chains) of the linking (Fab) antibody fragments of step 9 to the carboxy-terminal ends of the foreign class I MHC molecules of step 8; using SPPS technique.
11) Joining the light chains (or the heavy chains) of the linking (Fab) antibody fragments included in the complexes prepared in step 7 with the heavy chains (or the light chains) of the linking (Fab) antibody fragments included in the complexes prepared in step 10 by disulfide bond formation using techniques well known to people experienced in the art, e.g. catalyses of the disulfide bond in the presence of low molecular weight thiol reagents in reduced and oxidized form, as described in Saxena et al., Biochemistry 9: 5015-5021 (1970), incorporated herein by reference in its entirety.

12) Sensitizing the mammal against the foreign class I MHC molecules provided in step 8 using well known immunization techniques, e.g., subcutaneous injection of the foreign MHC molecules, either alone or mixed with a suitable immune adjuvant.

13) Administering the compound protein molecules prepared in step 11 to the mammal via a suitable route.

The suitable route for administering the said compound protein molecules depends on the type of the pathogenic cell, so,
a) for cancerous cells the compound protein molecules are administered by either intravenous injection (to induce a cytotoxic cell-mediated immune response against any 2ry deposits spreading through the blood stream), by spraying into an open wound during an operation conducted to excise the main bulk of the tumor (to induce a cytotoxic cell-mediated immune response against any 2ry deposits present within the operation field, or spreading to nearby tissues through the lymphatic system), or preferably via both routes.
b) for virus-infected cells the compound protein molecules are administered by intravenous injection, preferably after temporary suppression of the reproduction cycle of the pathogenic virus by a suitable drug, to decrease the level of anti-viral antibodies formed within the blood of the mammal, which would otherwise compete with the administered compound protein molecules for binding with the antigens displayed by the virus-infected cells. Non limiting examples of such drugs are Nucleoside Reverse Transcriptase Inhibitors (NRTIs) and non-nucleoside reverse transcriptase inhibitors (NNRTIs), which are well known to people experienced in the art.

The present invention also provides compound protein molecules to be used for inducing a cytotoxic cell-mediated immune response in a mammal against a pathogenic cell displaying an antigen on its outer surface, which are prepared using the techniques referred to herein before.

In a preferred embodiment, the compound protein molecule comprises in sequence: 1) a foreign class I MHC molecule; 2) a linking (Fab) antibody fragment; 3) an alpha-helix shaped chain linker; and 4) a (Fab) fragment of an antibody specific against the antigen displayed on the outer surface of the pathogenic cell. In another preferred embodiment, the compound protein molecule comprises in sequence: 1) a foreign class I MHC molecule; 2) a linking (Fab) antibody fragment; 3) an alpha-helix shaped chain linker; 4) a (Fab′2) fragment of an antibody specific against the antigen displayed on the outer surface of the pathogenic cell; 5) an alpha-helix shaped chain linker; 6) a linking (Fab) antibody fragment; and 7) a foreign class I MHC molecule. Referring now to the drawings in general, the illustrations are for the purpose of describing a preferred embodiment of the invention and are not intended to limit the invention thereto.

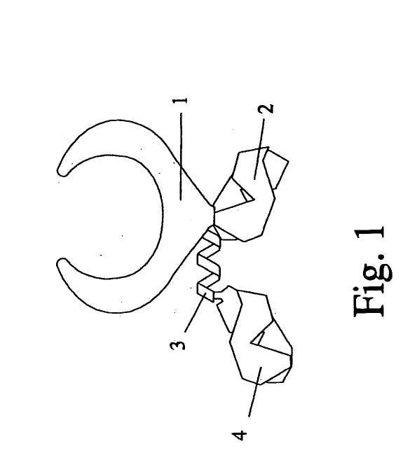

FIG. 1 is a schematic representation of a preferred embodiment of the compound protein molecules provided in the present invention, used to induce a cytotoxic cell-mediated immune response in a mammal against a pathogenic cell displaying an antigen on its outer surface, showing the orientation of its components relative to one another.

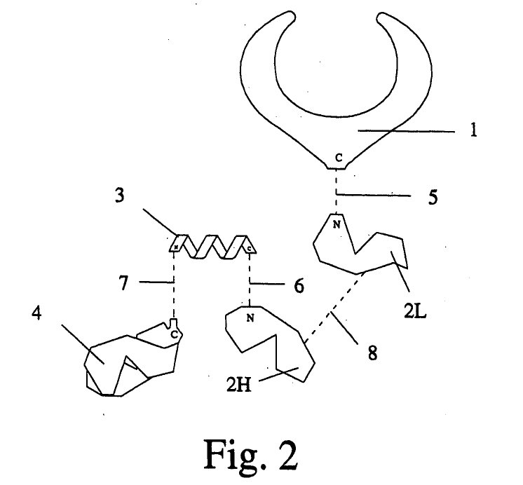

The main components of the compound protein molecule in this embodiment are a foreign class I MHC molecule (1); a linking (Fab) antibody fragment (2); an alpha-helix shaped chain linker (3); and a (Fab) fragment of an antibody (4) specific against the antigen displayed on the outer surface of the pathogenic cell. As shown in FIG. 2, and described herein before, the steps of preparing the compound protein molecule of FIG. 1 comprises:
a) Linking the amino-terminal end of the light chain of the linking (Fab) antibody fragment (2L) to the carboxy-terminal end of the foreign class I MHC molecule (1) through a peptide bond (5).
b) Linking the amino-terminal end of the heavy chain of the linking (Fab) antibody fragment (2H) to the carboxy-terminal end of the alpha-helix shaped chain linker (3) through a peptide bond (6).
c) Linking the amino-terminal end of the alpha-helix shaped chain linker (3) to the carboxy-terminal end of the heavy chain of the (Fab) antibody fragment (4) through a peptide bond (7), noting that the carboxy terminal end of the light chain of the (Fab) antibody fragment (4) had been blocked in an earlier step during the preparation, as described herein before.
d) Joining the light chain of the linking (Fab) antibody fragment (2L) and the heavy chain of the linking (Fab) antibody fragment (2H) by formation of a disulfide bond (8) in-between.

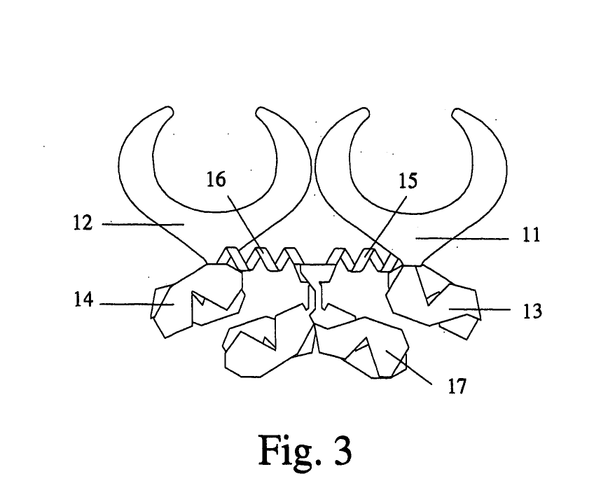

FIG. 3 is a schematic representation of another preferred embodiment of the compound protein molecules provided in the present invention, used to induce a cytotoxic cell-mediated immune response in a mammal against a pathogenic cell displaying an antigen on its outer surface, showing the orientation of its components relative to one another.

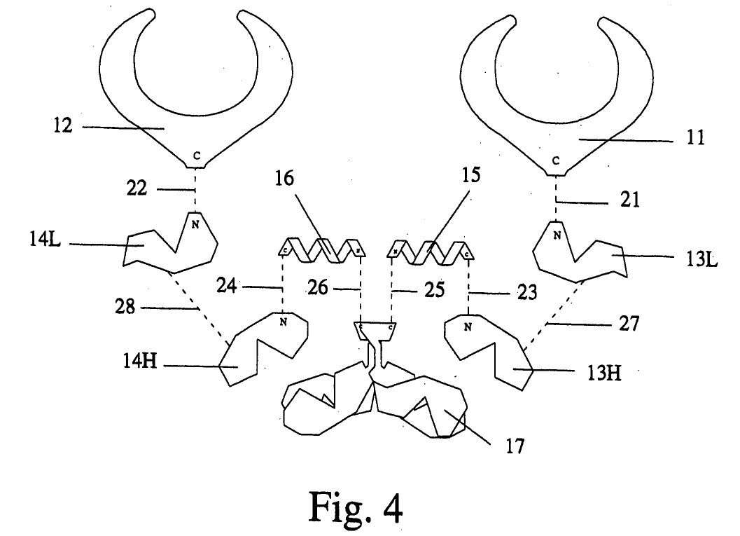

The main components of the compound protein molecule in this embodiment are two foreign class I MHC molecules (11,12); two linking (Fab) antibody fragments (13,14); two alpha-helix shaped chain linkers (15,16); and a (Fab′2) fragment of an antibody (17) specific against the antigen displayed on the outer surface of the pathogenic cell. As shown in FIG. 4, and described herein before, the steps of preparing the compound protein molecule of FIG. 3 comprises:
a) Linking the amino-terminal ends of the light chains of the linking (Fab) antibody fragments (13L,14L) to the carboxy-terminal ends of the foreign class I MHC molecules (11,12) through peptide bonds (21,22).
b) Linking the amino-terminal ends of the heavy chains of the linking (Fab) antibody fragments (13H,14H) to the carboxy-terminal ends of the alpha-helix shaped chain linkers (15,16) through peptide bonds (23,24).
c) Linking the amino-terminal ends of the alpha-helix shaped chain linkers (15,16) to the carboxy-terminal ends of the two heavy chains of the (Fab′2) antibody fragment (17) through peptide bonds (25,26), noting that the carboxy terminal ends of the two light chains of the (Fab′2) antibody fragment (17) had been blocked in an earlier step during the preparation, as described herein before.
d) Joining the light chains of the linking (Fab) antibody fragments (13L,14L) and the heavy chains of the linking (Fab) antibody fragments (13H,14H) by formation of disulfide bonds (27,28) in-between.

Certain modifications and improvements will occur to those skilled in the art upon a reading of the foregoing description. All modifications and improvements have been deleted herein for the sake of conciseness and readability but are properly within the scope of the following claims.

1. A method for inducing a cytotoxic cell-mediated immune response in a mammal against a pathogenic cell displaying an antigen on its outer surface, comprising the steps of:
a) specifying the type of the antigen displayed by the pathogenic cell;
b) providing a supply of monoclonal antibodies specific against the specified antigen;
c) blocking the carboxy-terminal ends of the light and heavy chains of the monoclonal antibodies provided in step (b), followed by digesting the monoclonal antibodies by Papain enzyme to yield (Fab) antibody fragments with blocked carboxy-terminal ends of their light chains;
d) providing a supply of alpha-helix shaped chain linkers;
e) linking the amino-terminal ends of the alpha-helix shaped chain linkers provided in step (d) to the carboxy-terminal ends of the heavy chains of the (Fab) antibody fragments provided in step (c), through peptide bonds;
f) providing a supply of light chains of linking (Fab) antibody fragments;
g) linking the amino-terminal ends of the light chains of the linking (Fab) antibody fragments provided in step (f) to the carboxy-terminal ends of the alpha-helix shaped chain linkers included in the complexes prepared in step (e), through peptide bonds;
h) providing a supply of foreign class I MHC molecules;
i) providing a supply of heavy chains of linking (Fab) antibody fragments;
j) linking the amino-terminal ends of the heavy chains of the linking (Fab) antibody fragments provided in step (i) to the carboxy-terminal ends of the foreign class I MHC molecules provided in step (h), through peptide bonds;
k) joining the light chains of the linking (Fab) antibody fragments included in the complexes prepared in step (g) with the heavy chains of the linking (Fab) antibody fragments included in the complexes prepared in step (j), through disulfide bonds;
l) sensitizing the mammal against the foreign class I MHC molecules provided in step (h); and
m) administering the compound protein molecules prepared in step (K) to the mammal.

2. The method of claim 1 wherein the mammal is a man.

3. The method of claim 1 wherein the pathogenic cell is a virus-infected cell.

4. The method of claim 3 wherein the administration of the compound protein molecules prepared in step (k) to the mammal is preceded by suppression of the reproduction cycle of the pathogenic virus.

5. The method of claim 1 wherein the pathogenic cell is a cancerous cell.

6. The method of claim 5 wherein the compound protein molecules prepared in step (k) are administered by spraying within an open wound during an operation conducted to excise the main bulk of the tumor.

7. The method of claim 1 wherein the compound protein molecules prepared in step (k) are administered by intravenous injection.

8. A method for inducing a cytotoxic cell-mediated immune response in a mammal against a pathogenic cell displaying an antigen on its outer surface, comprising the steps of:
a) specifying the type of the antigen displayed by the pathogenic cell;
b) providing a supply of monoclonal antibodies specific against the specified antigen;
c) blocking the carboxy-terminal ends of the light and heavy chains of the monoclonal antibodies provided in step (b), followed by digesting the monoclonal antibodies by Pepsin enzyme to yield (Fab′2) antibody fragments with blocked carboxy-terminal ends of their light chains;
d) providing a supply of alpha-helix shaped chain linkers;
e) linking the amino-terminal ends of the alpha-helix shaped chain linkers provided in step (d) to the carboxy-terminal ends of the heavy chains of the (Fab′2) antibody fragments provided in step (c), through peptide bonds;
f) providing a supply of light chains of linking (Fab) antibody fragments;
g) linking the amino-terminal ends of the light chains of the linking (Fab) antibody fragments provided in step (f) to the carboxy-terminal ends of the alpha-helix shaped chain linkers included in the complexes prepared in step (e), through peptide bonds;
h) providing a supply of foreign class I MHC molecules;
i) providing a supply of heavy chains of linking (Fab) antibody fragments;
j) linking the amino-terminal ends of the heavy chains of the linking (Fab) antibody fragments provided in step (i) to the carboxy-terminal ends of the foreign class I MHC molecules provided in step (h), through peptide bonds;
k) joining the light chains of the linking (Fab) antibody fragments included in the complexes prepared in step (g) with the heavy chains of the linking (Fab) antibody fragments included in the complexes prepared in step (j), through disulfide bonds;
l) sensitizing the mammal against the foreign MHC molecules provided in step (h); and
m) administering the compound protein molecules prepared in step (K) to the mammal.

9. The method of claim 8 wherein the mammal is a man.

10. The method of claim 8 wherein the pathogenic cell is a virus-infected cell.

11. The method of claim 10 wherein the administration of the compound protein molecules prepared in step (k) to the mammal is preceded by suppression of the reproduction cycle of the pathogenic virus.

12. The method of claim 8 wherein the pathogenic cell is a cancerous cell.

13. The method of claim 12 wherein the compound protein molecules prepared in step (k) are administered by spraying within an open wound during an operation conducted to excise the main bulk of the tumor.

14. The method of claim 8 wherein the compound protein molecules prepared in step (k) are administered by intravenous injection.

15. A method for inducing a cytotoxic cell-mediated immune response in a mammal against a pathogenic cell displaying an antigen on its outer surface, comprising the steps of:
a) specifying the type of the antigen displayed by the pathogenic cell;
b) providing a supply of monoclonal antibodies specific against the specified antigen;
c) blocking the carboxy-terminal ends of the light and heavy chains of the monoclonal antibodies provided in step (b), followed by digesting the monoclonal antibodies by Papain enzyme to yield (Fab) antibody fragments with blocked carboxy-terminal ends of their light chains;
d) providing a supply of alpha-helix shaped chain linkers;
e) linking the amino-terminal ends of the alpha-helix shaped chain linkers provided in step (d) to the carboxy-terminal ends of the heavy chains of the (Fab) antibody fragments provided in step (c), through peptide bonds;
f) providing a supply of heavy chains of linking (Fab) antibody fragments;
g) linking the amino-terminal ends of the heavy chains of the linking (Fab) antibody fragments provided in step (f) to the carboxy-terminal ends of the alpha-helix shaped chain linkers included in the complexes prepared in step (e), through peptide bonds;
h) providing a supply of foreign class I MHC molecules;
i) providing a supply of light chains of linking (Fab) antibody fragments;
j) linking the amino-terminal ends of the light chains of the linking (Fab) antibody fragments provided in step (i) to the carboxy-terminal ends of the foreign class I MHC molecules provided in step (h), through peptide bonds;
k) joining the heavy chains of the linking (Fab) antibody fragments included in the complexes prepared in step (g) with the light chains of the linking (Fab) antibody fragments included in the complexes prepared in step (j), through disulfide bonds;
l) sensitizing the mammal against the foreign class I MHC molecules provided in step (h); and
m) administering the compound protein molecules prepared in step (K) to the mammal.

16. The method of claim 15 wherein the mammal is a man.

17. The method of claim 15 wherein the pathogenic cell is a virus-infected cell.

18. The method of claim 17 wherein the administration of the compound protein molecules prepared in step (k) to the mammal is preceded by suppression of the reproduction cycle of the pathogenic virus.

19. The method of claim 15 wherein the pathogenic cell is a cancerous cell.

20. The method of claim 19 wherein the compound protein molecules prepared in step (k) are administered by spraying within an open wound during an operation conducted to excise the main bulk of the tumor.

21. The method of claim 15 wherein the compound protein molecules prepared in step (k) are administered by intravenous injection.

22. A method for inducing a cytotoxic cell-mediated immune response in a mammal against a pathogenic cell displaying an antigen on its outer surface, comprising the steps of:
a) specifying the type of the antigen displayed by the pathogenic cell;
b) providing a supply of monoclonal antibodies specific against the specified antigen;
c) blocking the carboxy-terminal ends of the light and heavy chains of the monoclonal antibodies provided in step (b), followed by digesting the monoclonal antibodies by Pepsin enzyme to yield (Fab′2) antibody fragments with blocked carboxy-terminal ends of their light chains;
d) providing a supply of alpha-helix shaped chain linkers;
e) linking the amino-terminal ends of the alpha-helix shaped chain linkers provided in step (d) to the carboxy-terminal ends of the heavy chains of the (Fab′2) antibody fragments provided in step (c), through peptide bonds;
f) providing a supply of heavy chains of linking (Fab) antibody fragments;
g) linking the amino-terminal ends of the heavy chains of the linking (Fab) antibody fragments provided in step (f) to the carboxy-terminal ends of the alpha-helix shaped chain linkers included in the complexes prepared in step (e), through peptide bonds;
h) providing a supply of foreign class I MHC molecules;
i) providing a supply of light chains of linking (Fab) antibody fragments;
j) linking the amino-terminal ends of the light chains of the linking (Fab) antibody fragments provided in step (i) to the carboxy-terminal ends of the foreign class I MHC molecules provided in step (h), through peptide bonds;
k) joining the heavy chains of the linking (Fab) antibody fragments included in the complexes prepared in step (g) with the light chains of the linking (Fab) antibody fragments included in the complexes prepared in step (j), through disulfide bonds;
l) sensitizing the mammal against the foreign class I MHC molecules provided in step (h); and
m) administering the compound protein molecules prepared in step (K) to the mammal.

23. The method of claim 22 wherein the mammal is a man.

24. The method of claim 22 wherein the pathogenic cell is a virus-infected cell.

25. The method of claim 24 wherein the administration of the compound protein molecules prepared in step (k) to the mammal is preceded by suppression of the reproduction cycle of the pathogenic virus.

26. The method of claim 22 wherein the pathogenic cell is a cancerous cell.

27. The method of claim 26 wherein the compound protein molecules prepared in step (k) are administered by spraying within an open wound during an operation conducted to excise the main bulk of the tumor.

28. The method of claim 22 wherein the compound protein molecules prepared in step (k) are administered by intravenous injection.

29. A compound protein molecule, used for inducing a cytotoxic cell-mediated immune response in a mammal against a pathogenic cell displaying an antigen on its outer surface, comprising:
1) a foreign class I MHC molecule, having an amino-terminal end and a carboxy-terminal end;
2) a (Fab) fragment of an antibody specific against the said antigen displayed on the outer surface of the pathogenic cell, and having an amino-terminal end and a carboxy-terminal end; and
3) intermediate linking means, by which the carboxy-terminal end of the said foreign class I MHC molecule is linked to the carboxy-terminal end of the said (Fab) antibody fragment.

30. A compound protein molecule, used for inducing a cytotoxic cell-mediated immune response in a mammal against a pathogenic cell displaying an antigen on its outer surface, comprising in sequence:
1) a foreign class I MHC molecule;
2) a linking (Fab) antibody fragment;
3) an alpha-helix shaped chain linker; and
4) a (Fab) fragment of an antibody specific against the said antigen displayed on the outer surface of the pathogenic cell.

31. A compound protein molecule, used for inducing a cytotoxic cell-mediated immune response in a mammal against a pathogenic cell displaying an antigen on its outer surface, comprising:
1) two foreign class I MHC molecules, each having an amino-terminal end and a carboxy-terminal end;
2) a (Fab′2) fragment of an antibody specific against the said antigen displayed on the outer surface of the pathogenic cell, and having two amino-terminal ends and two carboxy-terminal ends; and
3) intermediate linking means, by which the carboxy-terminal ends of the said foreign class I MHC molecules are linked to the carboxy-terminal ends of the said (Fab′2) antibody fragment.

32. A compound protein molecule, used for inducing a cytotoxic cell-mediated immune response in a mammal against a pathogenic cell displaying an antigen on its outer surface, comprising in sequence:
1) a foreign class I MHC molecule;
2) a linking (Fab) antibody fragment;
3) an alpha-helix shaped chain linker;
4) a (Fab′2) fragment of an antibody specific against the said antigen displayed on the outer surface of the pathogenic cell;
5) an alpha-helix shaped chain linker;
6) a linking (Fab) antibody fragment; and
7) a foreign class I MHC molecule.
